(19) United States
(12) Patent Application Publication
(10) Pub. No.: US 2019/0282189 A1
(43) Pub. Date: Sep. 19, 2019

(54) LIMITING IMAGING RADIATION DOSE AND IMPROVING IMAGE QUALITY DURING TREATMENT DELIVERY

(71) Applicant: Accuray Incorporated, Sunnyvale, CA (US)

(72) Inventors: Matthew Beneke, Madison, WI (US); Petr Jordan, Redwood City, CA (US); Trevor Laing, JR., San Jose, CA (US); Calvin R. Maurer, JR., San Jose, CA (US)

(21) Appl. No.: 15/922,688

(22) Filed: Mar. 15, 2018

(51) Int. Cl. A61B 6/00; A61B 6/06

(52) U.S. Cl. CPC A61B 6/4028; A61B 6/469; A61B 6/06; A61B 6/4452

(57) ABSTRACT

A method including imaging a first field of view (FOV) of a volume of interest (VOI) that includes a region of interest (ROI) from a first position and imaging the first FOV of the VOI from a second position. The method including receiving a first identification of a first portion of the imaged VOI designating the ROI to be imaged and a second identification of a second portion of the imaged VOI from the second position designating the ROI to be imaged. In response to the first identification, adjusting an aperture of a collimator of an imaging source to a second FOV corresponding to the ROI from the first position and imaging the ROI using the second FOV. In response to the second identification, adjusting the aperture of the collimator to a third FOV corresponding to the ROI from the second position and imaging the ROI using the third FOV.

### TECHNICAL FIELD

Embodiments of the disclosure relate to the field of radiation treatment delivery imaging.

### BACKGROUND

The intra-fraction motion of tumors during radiation therapy treatments is a concern in the modern era of image-guided radiotherapy. Providers of radiation oncology treatment systems have incorporated a kV x-ray imaging systems to obtain 2D radiographs of the radiation target, providing information about tumor position in real-time. This information can then be used by the treatment system to modify the delivery of therapeutic dose and compensate for the tumor motion.

In parallel to these developments, the radiation dose absorbed by patients from medical imaging procedures has come under scrutiny. The use of x-ray imaging system in radiation treatment procedures adds to the radiation dose absorbed by patients.

### SUMMARY OF EMBODIMENTS

In one embodiment, an apparatus comprises an x-ray imaging source of a helical delivery system, wherein the x-ray imaging source comprises a variable aperture collimator. The collimator may comprise a multi-leaf collimator. Alternatively, the collimator may comprise an iris collimator.

In one embodiment, a method comprises receiving an image from a treatment planning system comprising a volume of interest (VOI) comprising a region of interest (ROI) of a patient and, in response to receiving the image from the treatment planning system, adjusting an aperture of a collimator of an x-ray imaging source to a first field of view (FOV) corresponding to the VOI. The method also comprises imaging the first FOV of the VOI comprising the ROI of the patient with the x-ray imager. The method also comprises receiving an identification of the imaged VOI designating the ROI to be imaged and, in response to receiving the identification, adjusting the aperture of the collimator of the x-ray imaging source to a second FOV corresponding to the ROI, wherein the second FOV is different than the first FOV. The method also comprises imaging the ROI of the patient using the second FOV. The method may further comprise receiving a set up image comprising the VOI comprising the ROI of the patient and in response to receiving the set up image, adjusting the aperture of the collimator of the x-ray imaging source to a third FOV corresponding to the portion of the VOI, and imaging the third FOV of the portion of the VOI comprising the ROI of the patient.

In one embodiment, the method comprises generating a pre-diagnostic (or pre-setup) scan of a volume of interest (VOI) comprising a region of interest (ROI) of a patient and, in response to receiving the pre-diagnostic scan, adjusting an aperture of a collimator of an x-ray imaging source to a first field of view (FOV) corresponding to the VOI. The method also comprises imaging the first FOV of the VOI comprising the ROI of the patient with the x-ray imager and receiving a second identification of a second portion of the imaged first portion of the VOI designating the ROI to be imaged. In response to receiving the second identification, the method also comprises adjusting the aperture of the collimator of the x-ray imaging source to a second FOV corresponding to the ROI, wherein the second FOV is different than the first FOV, and imaging the ROI of the patient using the second FOV.

In one embodiment, the method comprises imaging a first field of view (FOV) of a volume of interest (VOI) comprising a region of interest (ROI) of a patient from a first position with a first x-ray imager comprising a first x-ray imaging source and imaging a second FOV of the VOI comprising the ROI of the patient from a second position with a second x-ray imager comprising a second x-ray imaging source. The method also comprises receiving a first identification of a first portion of the imaged VOI from the first position designating the ROI to be imaged and receiving a second identification of a second portion of the imaged VOI from the second position designating the ROI to be imaged. The method also comprises in response to receiving the first identification, adjusting an aperture of a collimator of the first x-ray imaging source to a third FOV corresponding to the ROI from the first position, the third FOV being different than the first FOV from the first position and imaging the ROI of the patient from the first position using the third FOV. The method also comprises, in response to receiving the second identification, adjusting the aperture of the collimator of the second x-ray imaging source to a fourth FOV corresponding to the ROI from the second position, the fourth FOV being different than the second FOV from the second position, and imaging the ROI of the patient from the second position using the fourth FOV.

### BRIEF DESCRIPTION OF THE DRAWINGS

Embodiments of the present disclosure are illustrated by way of example, and not by way of limitation, in the figures of the accompanying drawings.

FIG. 1 illustrates a helical delivery system in accordance with embodiments of the present disclosure.

FIG. 2 is a cross-section of the treatment imaging system of FIG. 1.

FIG. 3 is a flowchart illustrating a method for adjusting the aperture of a collimator of a kV imaging source to image a region of interest in accordance with embodiments of the present disclosure.

FIG. 4 is an illustration of generating an x-ray image having a full field of view (FOV) from a first angle.

FIG. 5 is an illustration of generating an x-ray image having a full FOV from a second angle.

FIG. 6A is an illustration of the identification of a region of interest (ROI) in the x-ray image generated in FIG. 4.

FIG. 6B is an illustration of the identification of a ROI in the x-ray image generated in FIG. 5.

FIG. 7 is an illustration of the collimator of FIG. 1 according to embodiments of the present disclosure.

FIG. 8 is an illustration of generating an x-ray image having a partial FOV from the first angle.

FIG. 9 is an illustration of generating an x-ray image having a partial FOV from the second angle.

FIG. 10 is an illustration of a method of adjusting the aperture of the collimator of the kV imaging source while the kV imaging source rotates from the first angle to the second angle.

FIG. 11A is an illustration of the x-ray image from the first angle generated in FIG. 8.

FIG. 11B is an illustration of the x-ray image from the second angle generated in FIG. 9.

FIG. 12 illustrates the configuration of an image-guided radiation treatment system in which embodiments of the present disclosure may be practiced.

FIG. 13 illustrates the configuration of gantry based radiotherapy system in which embodiments of the present disclosure may be practiced.

FIG. 14 illustrates the configuration of a portal imaging system in which embodiments of the present disclosure may be practiced.

### DETAILED DESCRIPTION

In order to control a radiation therapy procedure, the location of a target region (e.g., tumor) that is to be treated may be tracked with the use of a kilovoltage (kV) x-ray imaging system. This can be particularly important for targets that have the potential to move during treatment, such as target regions on or near a lung, on or near a heart, on or near a prostate, and so on. For some types of target regions, existing techniques of tracking the target regions may not be optimal due to exposure of a patient to additional radiation. For example, some target regions may require an increase in the energy of the imaging x-ray beam in order to get a useable x-ray image which exposes the patient to increased amounts of radiation. In another example, to accurately treat a target region that is moving within a patient, x-ray images of the target region may be generated at a sufficient frequency to verify the location of the target region, exposing the patient to additional radiation. Embodiments of the present disclosure relate to methods and systems for generating and using images of a smaller region of interest (ROI) within a treatment volume of interest (VOI) of a patient during radiation treatment delivery to minimize the patient's exposure to additional radiation.

During treatment delivery, dynamic tracking of the ROI may be performed based on the use of x-ray images taken to identify a target region. Once the location of the target region has been computed, the treatment delivery (e.g., radiation beam source position of the radiation treatment delivery system) may be adjusted to compensate for the dynamic motion of the target. In order to accurately track the motion of the target region, an x-ray imaging system may be used to continuously generate x-ray images of the target within a patient throughout the treatment. However, a result may be exposure of the patient to additional radiation.

An embodiment of the present disclosure may minimize the exposure to additional radiation issue described above by adjusting the field of view (FOV) of the kV imaging source with a collimator to a smaller field of view for a more specific region of interest (ROI) around the treatment target. Based on an identification of the ROI in, for example, a full FOV of the x-ray imaging system, an aperture of a collimator of the kV imaging source may be adjusted to correspond to the size or shape of the ROI, resulting in x-ray images of the ROI that may have a smaller FOV than the full FOV x-ray images generated during, for example, a patient set up stage. This may allow for the generation of x-ray images of the ROI having the same, or better, quality as the x-ray images with the full FOV without exposing the healthy tissue surrounding the ROI to unwanted radiation and decreasing the overall image radiation dose to the patient. In other words, embodiments of the present disclosure may reduce the amount of tissue being irradiate and, thereby, reduce the scatter and thus improve the image quality of the reduced FOV being imaged versus the image quality of that region with a larger FOV (e.g., uncollimated).

It should be noted that in some embodiments described below, the same radiation source may be used to provide both higher energy therapeutic radiation and lower energy imaging radiation. In such an embodiment, the radiation source may be a linear acceleration (LINAC) having a primary collimator and a secondary collimator. The primary collimator may have a fixed aperture and is positioned after the electron beam has passed through the x-ray emitting target. The secondary collimator may be positioned after the primary collimator and may have a variable aperture, as will be discussed in more detail below.

Embodiments of the disclosure are particularly applicable to a radiosurgical treatment system and method and it is in this context that these embodiments of the disclosure will be described. It will be appreciated, however, that the system and method in accordance with the disclosure has greater utility, such as to other types of treatments wherein it is necessary to accurately position treatment at a target region within the patient in order to avoid damaging healthy tissue such as to other types of medical procedures with other types of medical instruments.

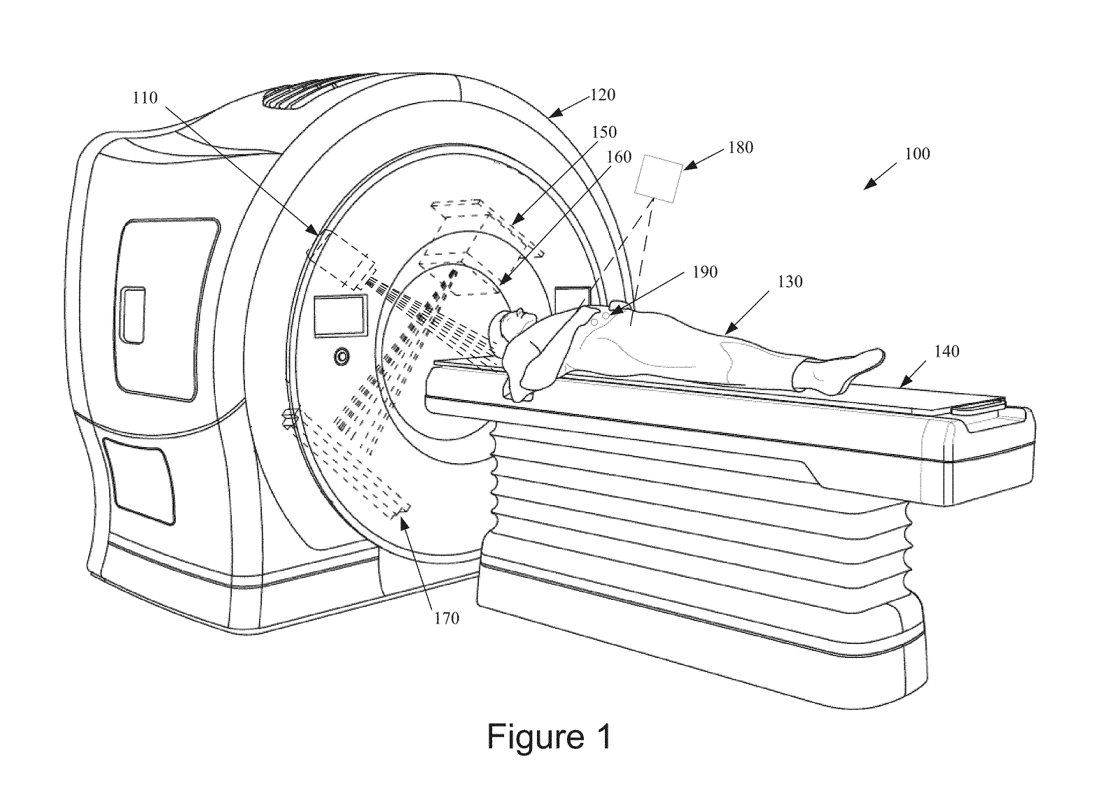

FIG. 1 illustrates a helical delivery system 100 in accordance with embodiments of the present disclosure. The helical delivery system 100 of FIG. 1 may include a linear accelerator (LINAC) 110 mounted to a ring gantry 120. The LINAC 110 may be used to generate a narrow intensity modulated pencil beam (i.e., treatment beam) by directing an electron beam towards an x-ray emitting target. The treatment beam may deliver radiation to a target region (i.e., a tumor). The ring gantry 120 generally has a toroidal shape in which the patient 130 extends through a bore of the ring/toroid and the LINAC 110 is mounted on the perimeter of the ring and rotates about the axis passing through the center to irradiate a target region with beams delivered from one or more angles around the patient. During treatment, the patient 130 may be simultaneously moved through the bore of the gantry on treatment couch 140.

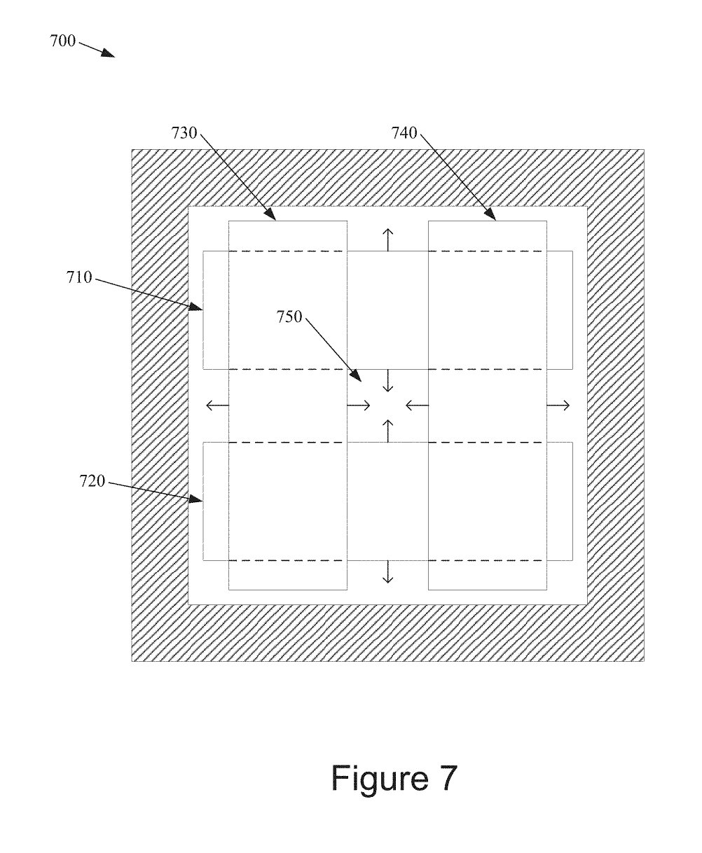

The helical delivery system 100 of FIG. 1 includes a treatment imaging system, which may include a kV imaging source 150 and an x-ray detector 170. The kV imaging source 150 may be used to generate x-ray images of a ROI of patient 130 by directing a sequence of x-ray beams at the ROI which are incident on the x-ray detector 170 opposite the kV imaging source 150 to image the patient 130 for setup and generate in-treatment images. The treatment imaging system may further include a collimator 160. In one embodiment, the collimator 160 may be a variable aperture collimator as discussed in FIG. 7. In another embodiment, the collimator 160 may be a multi-leaf collimator (MLC). The MLC includes a housing that houses multiple leaves that are movable to adjust an aperture of the MLC to enable shaping of an imaging x-ray beam. In another embodiment, the variable aperture collimator 160 may be an iris collimator containing trapezoidal blocks that move along a frame in a manner similar to a camera iris to produce an aperture of variable size that enables shaping of the imaging x-ray beam. The kV imaging source 150 and the x-ray detector 170 may be mounted orthogonally relative to the LINAC 110 (e.g., separated by 90 degrees) on the ring gantry 120 and may be aligned to project an imaging x-ray beam at a target region and to illuminate an imaging plane of detector 170 after passing through the patient 130. In some embodiments, the LINAC 110 and/or the kV imaging source 150 may be mounted to a C-arm gantry in a cantilever-like manner, which rotates the LINAC 110 and kV imaging source 150 about the axis passing through the isocenter.

In one embodiment, the radiation therapy system 100 may include a motion detection device 180 to determine target motion. The motion detecting device 180 may detect external patient motion (such as chest movement during respiration) that occurs within an imaging field. The motion detecting device 180 can be any sensor or other device capable of identifying target movement. The motion detecting device 180 may be, for example, an optical sensor such as a camera, a pressure sensor, an electromagnetic sensor, or some other sensor that can provide motion detection without delivering ionizing radiation to a patient 130 (e.g., a sensor other than an x-ray imaging system). In one embodiment, the motion detecting device 180 acquires measurement data indicative of target motion in real-time. Alternatively, the measurement data may be acquired at a frequency that is higher (potentially substantially higher) than can be achieved or than is desirable with x-ray imaging (due to ionizing radiation delivered to the patient 130 with each x-ray image). In one embodiment, the motion detecting device 180 does not provide a high absolute position accuracy. Instead, the motion detecting device 180 may provide sufficient relative position accuracy to detect patient movement and/or target movement.

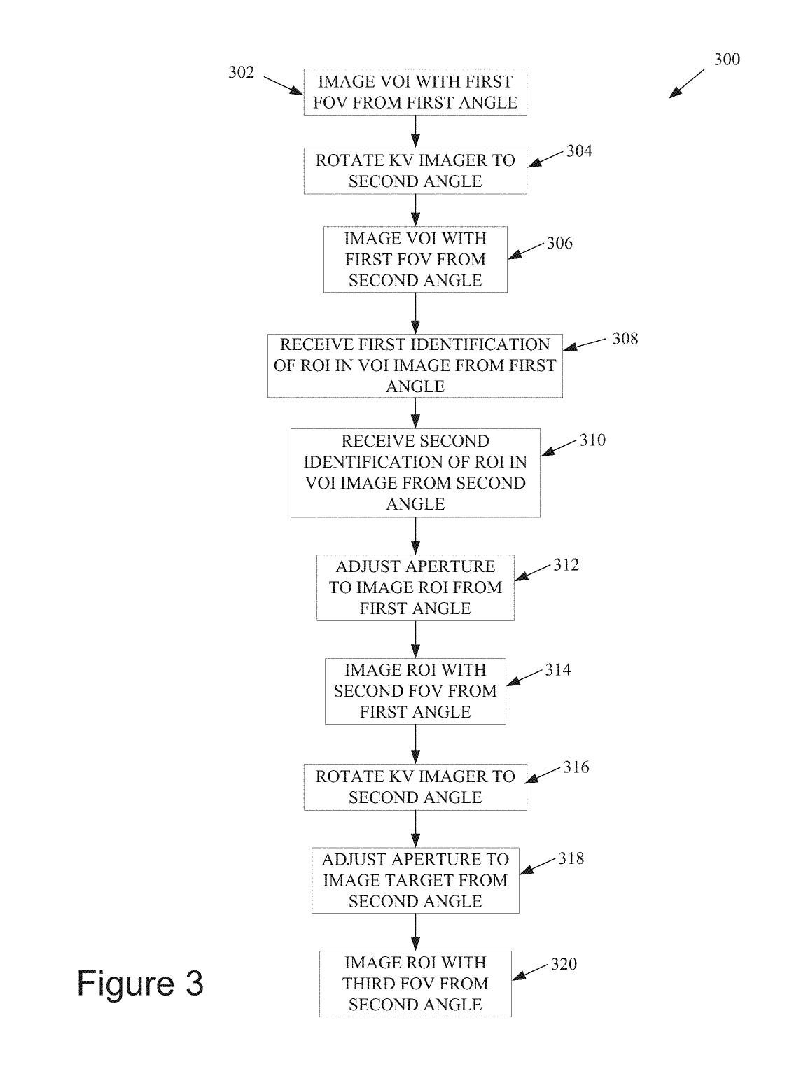

In one embodiment, the motion detecting device 180 is an optical system, such as a camera. The optical system may track the position of light-emitting diodes (LEDs) 190 situated on patient 130. Alternatively, the optical system may directly track a surface region (e.g., skin surface) of patient 130, as distinguished from tracking LEDs 190 on the patient 130. There may be a correlation between movement of a target region, described in more detail at FIGS. 3-6 below, and movement of the LEDs and/or surface region of the patient 130. A correlation model may be generated between the position of the target region and the tracking LEDs 190 and/or surface region of the patient 130 to predict the position of the target region at a later time. Based on the prediction, the aperture of the collimator 160 of the kV imaging source 150 may be adjusted to correspond to the predicted position of the target region. Adjustment of the aperture of the collimator 160 will be described in more detail at FIG. 7 below.

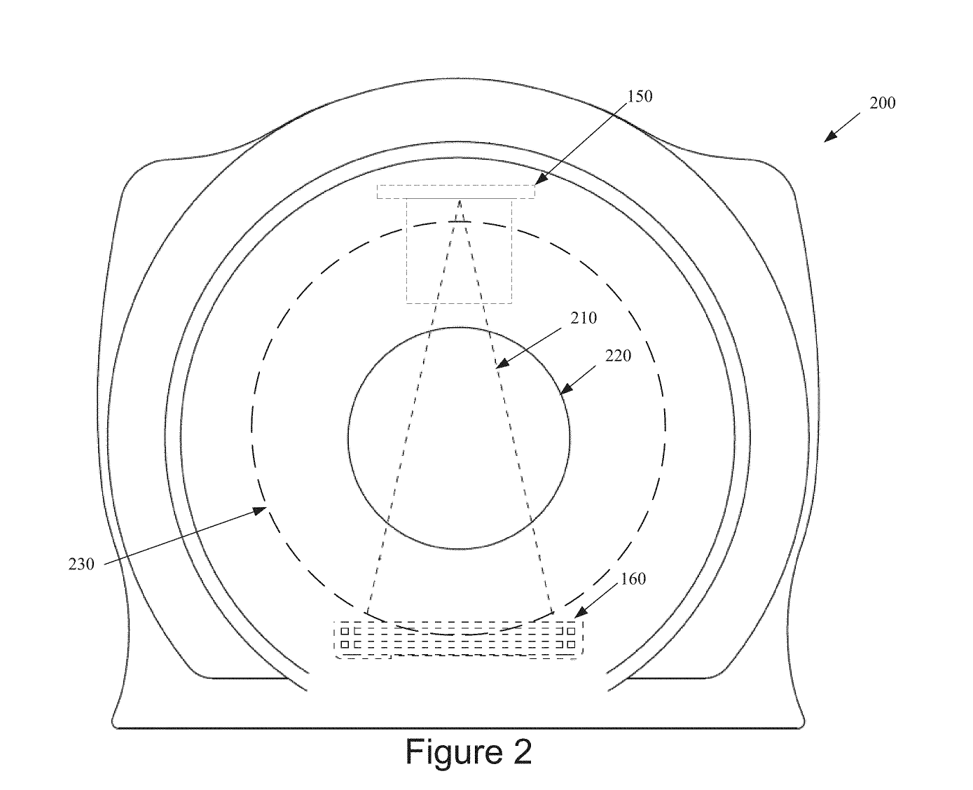

FIG. 2 is a cross-section 200 of the treatment imaging system of FIG. 1. As previously discussed, the kV imaging source 150 projects an imaging x-ray beam 210 through the bore 220 of the treatment system which illuminates the imaging plane of x-ray detector 170 after passing through a patient. The kV imaging source 150 and x-ray detector 170 may rotate along a circular track 230 of the ring gantry 120 around the bore 220 of the treatment system to generate x-ray images of a target region from multiple angles.

FIG. 3 is a flowchart 300 illustrating a method of adjusting the aperture of a collimator of a kV imaging source to image a target region in accordance with embodiments of the present disclosure. The method of FIG. 3 allows for the identification of a ROI including a target region from a full FOV image of a VOL Using the identification, the treatment imaging system may adjust the aperture of the collimator 160 to correspond to the identified ROI, minimizing radiation exposure of tissue surrounding the ROI. At block 302, a kV imaging source 150 and an x-ray detector 170 generate an image of a VOI of a patient that contains a target region from a first angle. At block 304, after generating an x-ray image of the VOI of the patient from the first angle, the kV imaging source 150 and x-ray detector 170 may rotate to a second angle. At block 306, the kV imaging source 150 and x-ray detector 170 generate an image of the VOI of the patient that contains the target region from a second angle. In some embodiments, the first angle and second angle may be determined prior to treatment using a treatment planning system. The images of the VOI may be captured with a full FOV where the collimation device may not shape the imaging x-ray beam. In some embodiments, an ROI including the target region may be identified during a treatment planning phase and images of the VOI may be captured with a partial FOV corresponding to the ROI identified during treatment planning.

At block 308, the treatment imaging system receives an identification of a ROI that includes the target region in the VOI image generated at block 302. In some embodiments, the target region may be identified by a user selecting the target region from the generated image. In other embodiments, the target region may be identified by the treatment imaging system using image recognition software or the like. At block 310, the treatment imaging system receives an identification of a ROI that includes the target region in the VOI image generated at block 306. The target region may be identified using methods similar to those described at block 308. At block 312, the aperture of the collimator 160 of the kV imaging source 150 may be adjusted using the methods previously described to correspond to the ROI identified at block 308. The adjustment of the aperture may allow the kV imaging source 150 and x-ray detector 170 to generate images with a partial FOV that is smaller than the FOV at block 302. At block 314, the kV imaging source 150 and x-ray detector 170 generate an image with the partial FOV of the ROI from the first angle, where the resultant image may correspond to the ROI identified at block 308. At block 316, after generating an x-ray image of the ROI of the patient from the first angle, the kV imaging source 150 and x-ray detector 170 may rotate to the second angle. At block 318, the aperture of the collimator 160 of the kV imaging source 150 may be adjusted using the methods previously described to correspond to the ROI identified at block 310. The adjustment of the aperture may allow the kV imaging source 150 and x-ray detector 170 to generate images with a partial FOV that is smaller than the FOV at block 306. At block 320, the kV imaging source 150 and x-ray detector 170 generate an image with the partial FOV of the ROI from the second angle, where the resultant image may correspond to the ROI identified at block 310. The present embodiment describes imaging an ROI with a partial FOV to reduce the radiation dose delivered to patient 130. However, the actual tracking of a target region may be used in conjunction with other techniques using external markers attached to the patient 130 to correlate movement of the external markers to movement of the target region. An example of one such system is the Synchrony™ respiratory tracking system developed by Accuray, Inc.

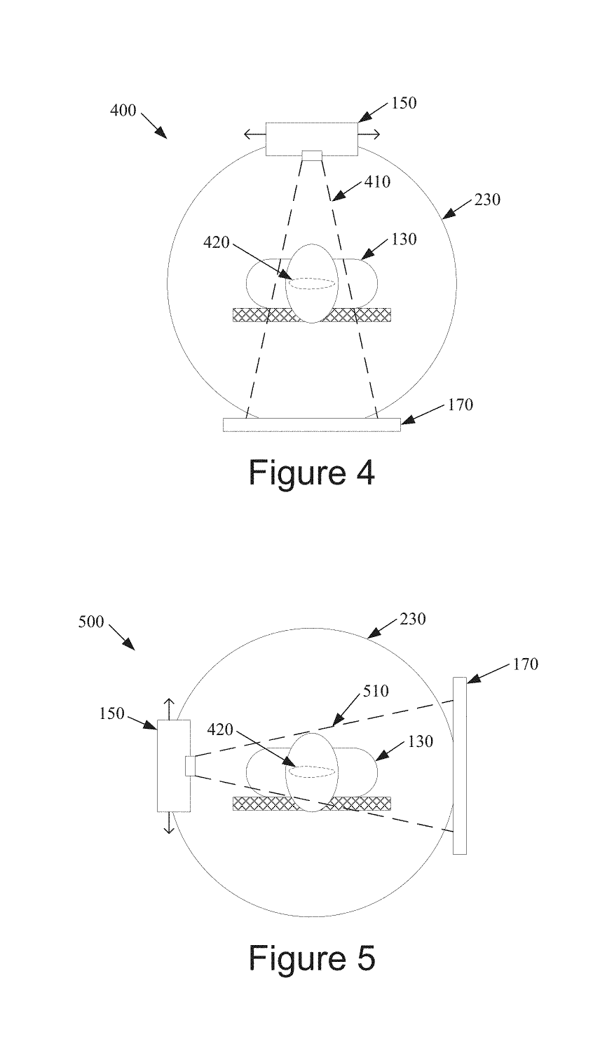

FIG. 4 is an illustration 400 of generating an x-ray image having a full FOV from the first angle. The illustration shown in FIG. 4 may be representative of block 302 of the method of FIG. 3 according to embodiments of the present disclosure. The kV imaging source 150 and the x-ray detector 170 generate an imaging x-ray beam 410 having a full FOV of a VOI of the patient 130 that contains a target region 420 from a first angle. The imaging x-ray beam 410 passes through the patient 130 and the target region 420 and is incident on the x-ray detector 170. The imaging x-ray beam 410 illuminates an imaging plane of the x-ray detector 170 after passing through the patient 130, which may be used by the treatment imaging system to generate an x-ray image of the VOI containing the target region 420 at a first position from the first angle. Once the x-ray image of the VOI from the first angle is acquired, the kV imaging source 150 and the x-ray detector 170 may rotate along the circular track 230 to a second angle as previously discussed at block 304.

Once the kV imaging source 150 and the x-ray detector 170 arrive at the second angle position, an image covering the full FOV of the VOI may be generated. The illustration 500 shown in FIG. 5 may be representative of block 306 of the method of FIG. 3 according to embodiments of the present disclosure. The kV imaging source 150 and the x-ray detector 170 generate an imaging x-ray beam 510 having a full FOV of a VOI of the patient 130 that contains a target region 420 from a second angle. The imaging x-ray beam 510 passes through the patient 130 and the target region 420 and are incident on the x-ray detector 170. The imaging x-ray beam 510 illuminates an imaging plane of the x-ray detector 170 after passing through the patient 130, which may be used by the treatment imaging system to generate an x-ray image of the VOI containing the target region 420 from the second angle.

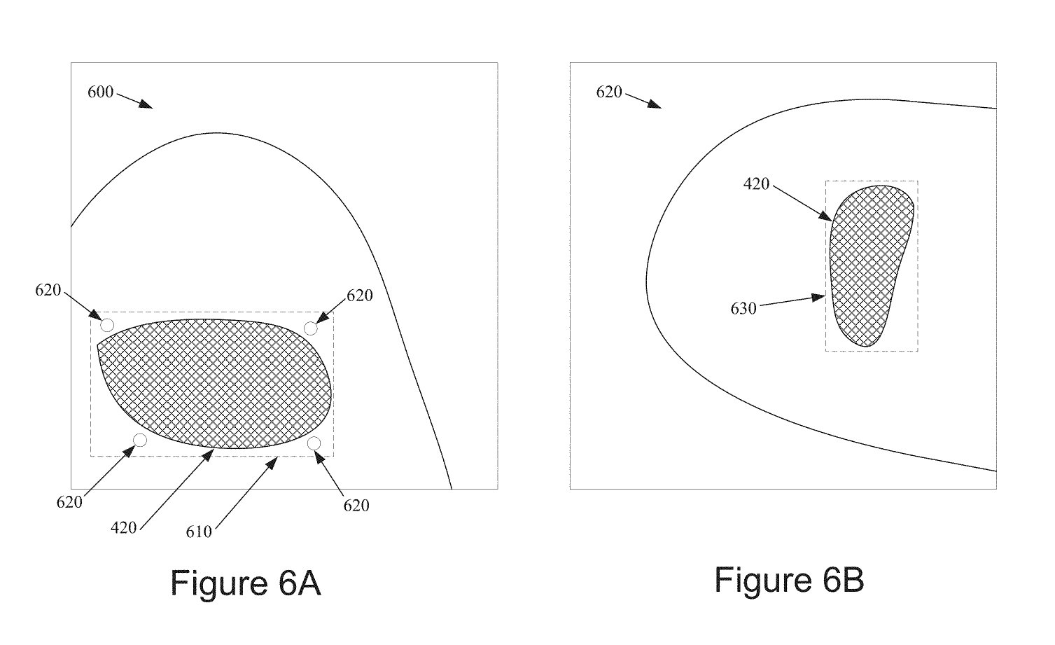

FIG. 6A is an illustration of the identification of a region of interest (ROI) in the x-ray image generated in FIG. 4. The generated x-ray image 600 of the VOI from the first angle may include the target region 420 and internal reference structures 620 located near the target region 420. In the illustrated embodiment, the internal reference structures 620 may be fiducial markers that are implanted in or near the target region 420 and that are visible in the x-ray image 600. In another embodiment, the internal reference structure 620 may be a bone of patient 130 located in close proximity to the target region 420. In some embodiments, it may be possible to generate a visible x-ray image of the target region 420 and direct target imaging of the target region 420 may be performed. The treatment system may then receive an identification of a ROI 610 from the first angle that includes the target region 420. As previously described, in some embodiments the identification of the ROI may be based on a selection of the ROI by a user. Selection of the ROI by the user may be completed by the user selecting the ROI from a user interface of the helical delivery system. In other embodiments, the ROI may be identified automatically by the helical delivery system using image recognition software or the like. In other embodiments, selection of the ROI may be received from a treatment planning system or a prediagnostic scan.

FIG. 6B is an illustration of the identification of a ROI in the x-ray image generated in FIG. 5. The generated x-ray image 620 of the VOI from the second angle may contain the target region 420 as well as the VOI surrounding the target region. The treatment system may then receive an identification of a ROI 630 from the second angle that includes the target region 420. Identification of the ROI 630 may be completed using methods similar to the methods described in FIG. 6A.

FIG. 7 is an illustration 700 of the collimator 160 of FIG. 1 according to embodiments of the present disclosure. In one embodiment, the collimator 160 may be a variable aperture collimator. The aperture 750 of the collimator 160 of the kV imaging source 150 may be adjusted as previously described at block 312 and block 318 of FIG. 3. The size and/or shape of the aperture may be adjusted to correspond to the ROI identified in FIGS. 6A and 6B. The collimator 160 may be composed of a series of plates 710, 720, 730, 740, where the positioning of the plates create aperture 750 in the center of the plates. An imaging x-ray beam passes through the aperture 750 and the shape of the imaging x-ray beam may correspond to the shape of the aperture 750. Therefore, the size and shape of the x-ray beam may be adjusted by changing the size and shape of the aperture 750 by moving plates 710, 720, 730, 740 within the collimator 160. The plates 710, 720, 730, 740 may be made of a material that prevents the passage radiation, such as lead. Plates 710 and 720 may be located on a parallel plane within the collimator 160 and may move vertically independent of one another along a vertical axis. Plates 730 and 740 may be located on a parallel plane within the collimator 160 and may move horizontally independent of one another along a horizontal axis. In some embodiments, plates 710, 720, 730, 740 may be moved by a series of actuators coupled to plates 710, 720, 730, 740 that may extend or retract. In another embodiment, plates 710, 720, 730, 740 may be moved manually by a user sliding the plates along the horizontal or vertical axes and securing plates 710, 720, 730, 740 in place with fasteners. Thus, the aperture 750 and the x-ray beam may be adjusted to various rectangular shapes having various sizes. As previously described, in some embodiments the collimator 160 may be a variable aperture collimator containing trapezoidal blocks that move along a frame in a manner similar to a camera iris to produce an aperture of variable size that enables shaping of the imaging x-ray beam.

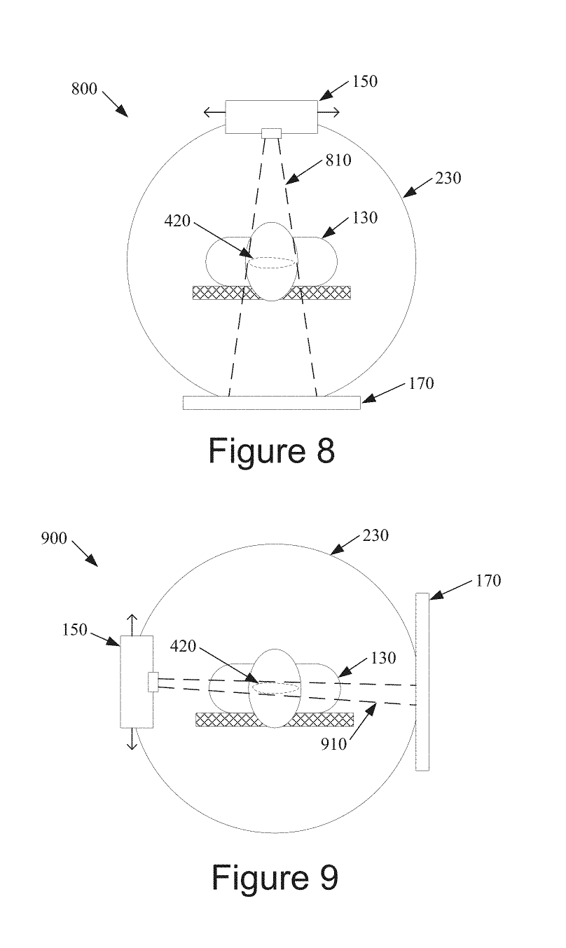

FIG. 8 is an illustration 800 of generating an x-ray image having a partial FOV from the first angle. The illustration shown in FIG. 8 may be representative of block 314 of the method of FIG. 3 according to embodiments of the present disclosure. Prior to imaging, the collimator 160 may adjust the aperture to correspond to the ROI identified in FIG. 6A using the methods described in FIG. 7, which may create a partial FOV for the kV imaging source 150. The kV imaging source 150 may then generate an imaging x-ray beam 810 with the partial FOV of the ROI of the patient 130 that includes the target region 420 from the first angle. The imaging x-ray beam 810 passes through the patient 130 and the target region 420 and incident on the x-ray detector 170. The imaging x-ray beam 810 illuminates an imaging plane of the x-ray detector 170 after passing through the patient 130, which may be used by the treatment imaging system to generate an x-ray image of the ROI including the target region 420 from the first angle. Once the x-ray image of the ROI from the first angle is acquired, the kV imaging source 150 and the x-ray detector 170 may rotate along the circular track 230 to a second angle.

Once the kV imaging source 150 and the x-ray detector 170 arrive at the second angle, an image covering the partial FOV of the ROI from the second angle may be generated. The illustration 900 shown in FIG. 9 may be representative of block 320 of the method of FIG. 3 according to embodiments of the present disclosure. Prior to imaging, the collimator 160 may adjust the aperture to correspond to the ROI identified in FIG. 6B using the methods described in FIG. 7, which may create a partial FOV for the kV imaging source 150. The kV imaging source 150 and the x-ray detector 170 may then generate an imaging x-ray beam 910 with the partial FOV of the ROI of the patient 130 that includes the target region 420 from the second angle. The imaging x-ray beam 910 passes through the patient 130 and the target region 420 and are collected by the x-ray detector 170. The imaging x-ray beam 810 illuminates an imaging plane of the x-ray detector 170 after passing through the patient 130, which may be used by the treatment imaging system to generate an x-ray image of the ROI containing the target region 420 from the first angle. Once the x-ray image of the ROI from the first angle is acquired, the kV imaging source 150 and the x-ray detector 170 may rotate along the circular track 230 and return to the first angle, where the imaging sequence may be repeated.

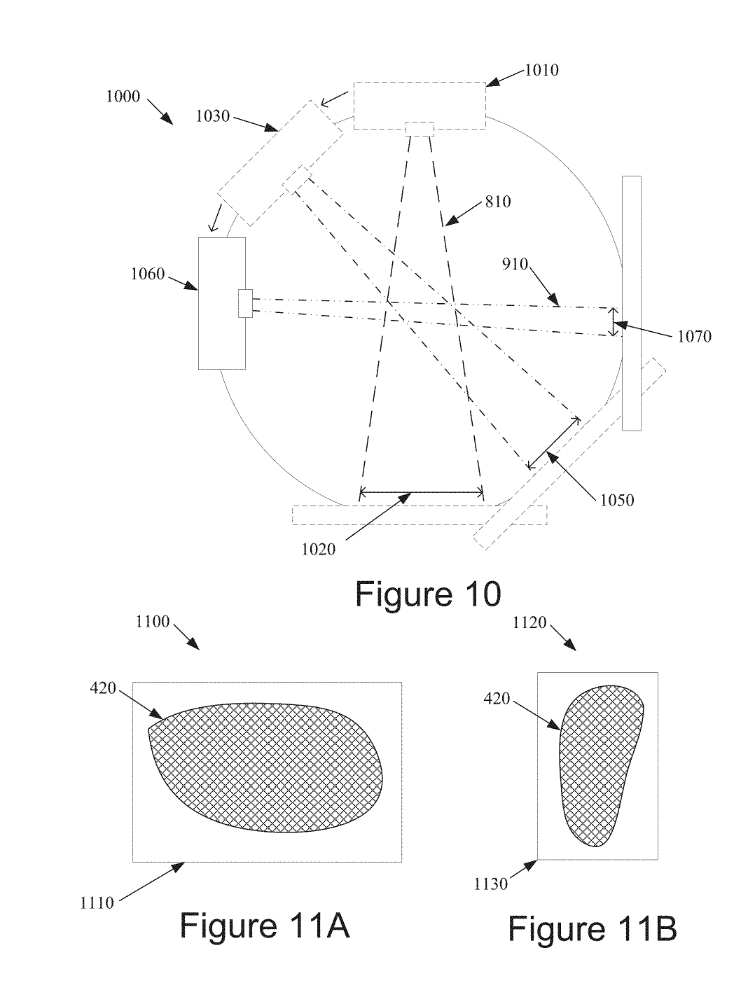

FIG. 10 is an illustration 1000 of a method of adjusting the aperture of the collimator of the kV imaging source 150 while the kV imaging source 150 rotates from the first angle to the second angle. The kV imaging source 150 may be located at the first angle 1010 and may generate an imaging x-ray beam 810 with a FOV 1020 to image the ROI as shown in FIG. 8. After the x-ray image has been captured, the kV imaging source 150 and x-ray detector 170 may begin to rotate to the second angle. As the kV imager rotates towards the second angle, the aperture of the collimator of the kV imaging source 150 may begin to adjust in accordance with the previously described methods. In one embodiment, at angle 1030 the kV imager may be in the process of rotating between the first angle and the second angle. The aperture of the collimator of the kV imaging source 150 may be adjusting, decreasing the FOV 1050 relative to the FOV 1020 at the first angle 1010. In another embodiment, the aperture of the collimator of the kV imaging source 150 may be adjusted once the kV imager has arrived at the second angle. When the kV imager arrives at the second angle 1060 the adjustment of the aperture of the collimator of the kV imaging source 150 to correspond to the ROI identified in FIG. 6B may be complete. The kV imager may then generate an imaging x-ray beam 910 with a FOV 1070 to image the ROI as shown in FIG. 9.

FIG. 11A is an illustration 1100 of the x-ray image from the first angle generated in FIG. 8. The x-ray image 1110 may correspond to the ROI identified in FIG. 6A and include the target region 420 of the patient 130 from the first angle. FIG. 11B is an illustration 1120 of the x-ray image from the second angle generated in FIG. 9. The x-ray image 1130 may correspond to the ROI identified in FIG. 6B and include the target region 420 of the patient 130 from the second angle. In some embodiments, the imaging sequence may be repeated during a treatment session using the ROI's identified in FIGS. 6A and 6B. In other embodiments, the treatment imaging system may receive an identification of a new ROI in the x-ray images of FIGS. 11A and 11B using the previously described methods and a new imaging sequence may begin.

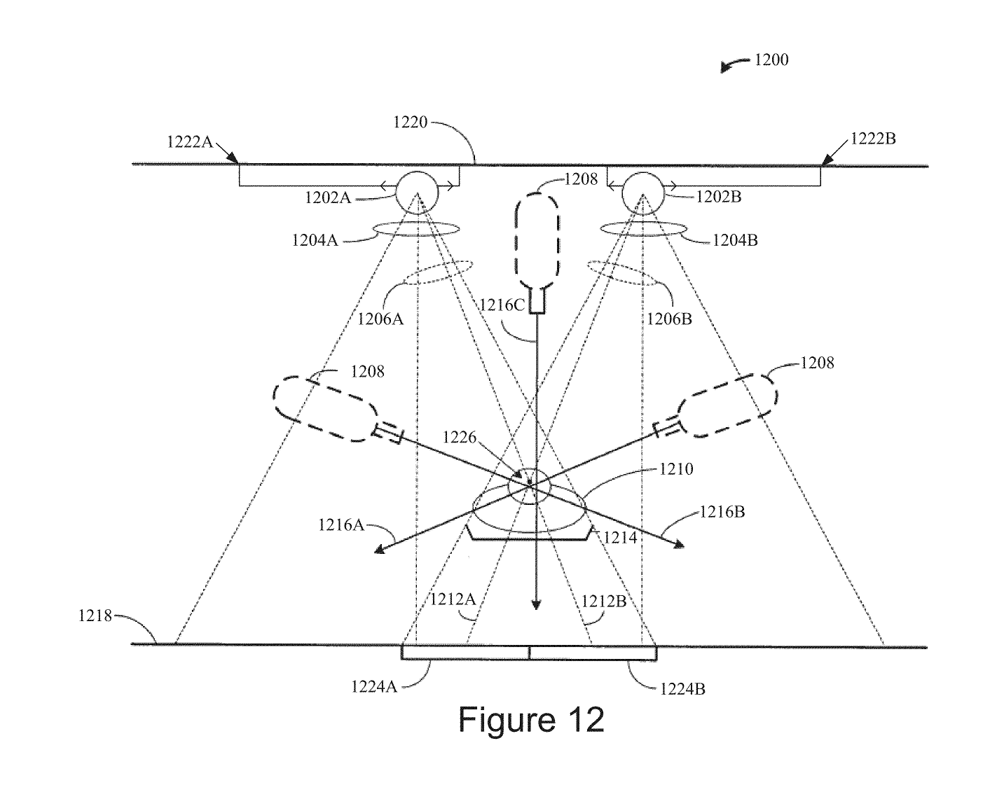

FIG. 12 illustrates the configuration of an image-guided radiation treatment (IGRT) system 1200 in which embodiments of the present disclosure may be practiced. The IGRT system 1200 may include to kV imaging sources 1202A and 1202B and may be mounted on tracks 1222A and 1222B on the ceiling 1220 of an operating room and may be aligned to project imaging x-ray beams 1204A and 1204B from two different positions such that a ray 1212A of beam 1204A intersects with a ray 1212B of beam 1204B at an imaging center 1226 (i.e., isocenter), which provides a reference point for positioning the LINAC 1208 to generate treatment beams 1216A, 1216B and 1216C and the patient 1210 on treatment couch 1214 during treatment. After passing through the patient 1210, imaging x-ray beams 1204A and 1204B may illuminate respective imaging surfaces of x-ray detectors 1224A and 1224B, which may be mounted at or near the floor 1218 of the operating room and substantially parallel to each other (e.g., within 5 degrees). The kV imaging sources 1202A and 1202B may be substantially coplanar such that the imaging surfaces of kV imaging sources 1202A and 1202B form a single imaging plane. In one embodiment, kV imaging sources 1202A and 1202B may be replaced with a single kV imaging source. Once an x-ray image of the patient 1214 has been generated, the LINAC 1208 may rotate to generate a treatment beam 1216 from a different angle. While the LINAC 1208 rotates to the different angle, the kV imaging sources 1202A and 1202B may move along tracks 1222A and 1222B to generate x-ray images of the patient 1210 from a new angle.

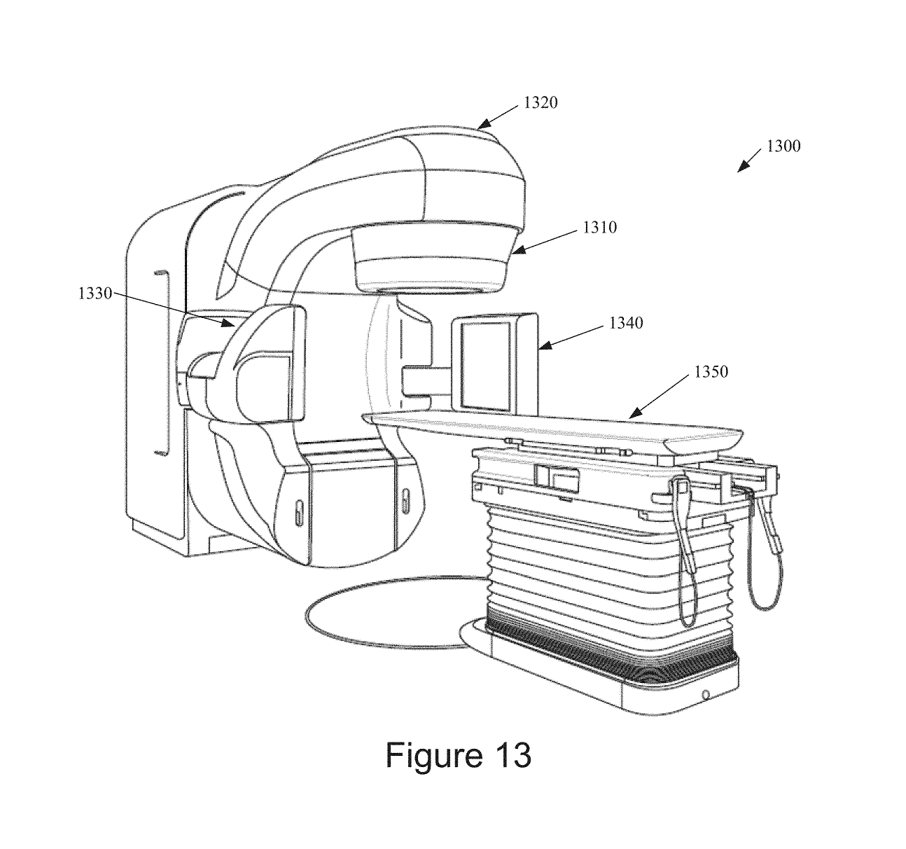

In some embodiments, the kV imaging source and LINAC may be used in other types of gantry-based systems, for example, the TrueBeam™ radiotherapy system manufactured by Varian Medical Systems as shown in FIG. 13. The radiotherapy system 1300 may include a LINAC 1310 mounted to a C-arm gantry 1320 that rotates about the axis passing through the isocenter. The radiotherapy system may also include a treatment imaging system comprised of a kV imaging source 1330 and an x-ray detector 1340. The kV imaging source 1330 having a variable aperture collimator and the x-ray detector 1340 may be mounted to robotic arms located opposite one another relative to the isocenter of the C-arm gantry 1320 to allow for imaging of a VOI of a patient on a treatment couch 1350.

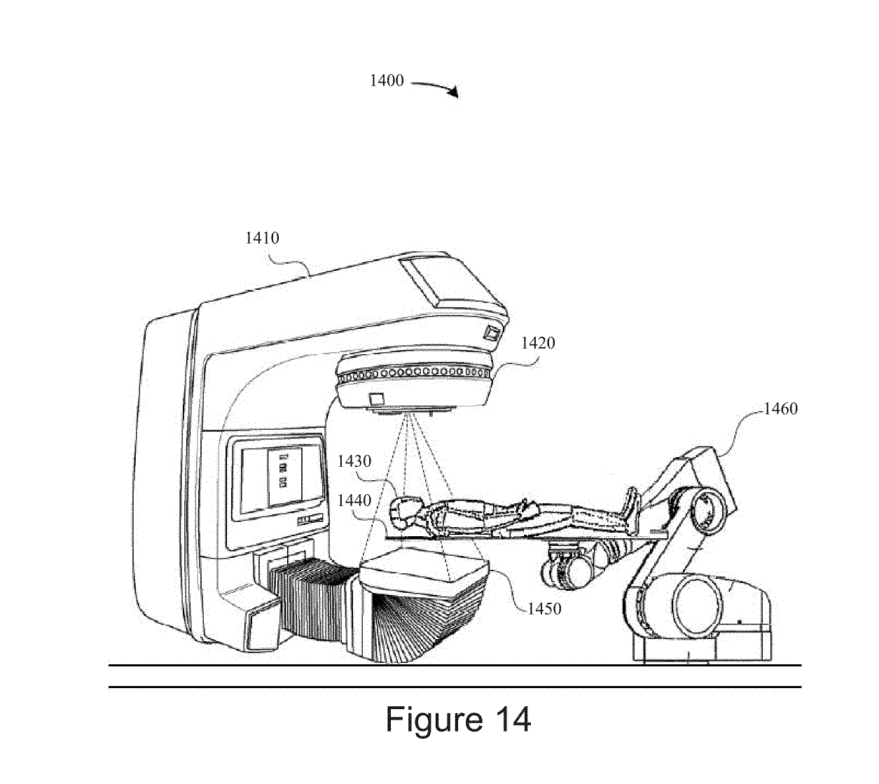

Embodiments of the present disclosure may be implemented in a portal imaging system 1400 as shown in FIG. 14. In the portal imaging system 1400 the beam energy of a LINAC may be adjusted during treatment and may allow the LINAC to be used for both x-ray imaging and radiation treatment. The gantry based radiation system 1400 includes a gantry 1410, a radiation source 1420 (i.e. a LINAC) and a portal imaging device 1450. The gantry 1410 may be rotated to an angle corresponding to a selected projection and used to acquire an x-ray image of a VOI of a patient 1430 on a treatment couch 1440. The radiation source 1420 may then generate an x-ray beam that passes through the VOI of the patient 1430 and are incident on the portal imaging device 1450, creating an x-ray image of the VOL After the x-ray image of the VOI has been generated, the beam energy of the radiation source 1420 may be increased so the radiation source 1420 may generate a treatment beam to treat a target region of the patient 1430.

Alternatively, the kV imaging source and methods of operations described herein may be used with yet other types of gantry-based systems. In some gantry-based systems, the gantry rotates the kV imaging source and LINAC around an axis passing through the isocenter. Gantry-based systems include ring gantries having generally toroidal shapes in which the patient's body extends through the bore of the ring/toroid, and the kV imaging source and LINAC are mounted on the perimeter of the ring and rotates about the axis passing through the isocenter. Gantry-based systems may further include C-arm gantries, in which the kV imaging source and LINAC are mounted, in a cantilever-like manner, over and rotates about the axis passing through the isocenter. In another embodiment, the kV imaging source and LINAC may be used in a robotic arm-based system, which includes a robotic arm to which the kV imaging source and LINAC are mounted.

Unless stated otherwise as apparent from the foregoing discussion, it will be appreciated that terms such as “processing,” “computing,” “generating,” “comparing” “determining,” “calculating,” “performing,” “identifying,” or the like may refer to the actions and processes of a computer system, or similar electronic computing device, that manipulates and transforms data represented as physical (e.g., electronic) quantities within the computer system's registers and memories into other data similarly represented as physical within the computer system memories or registers or other such information storage or display devices. Embodiments of the methods described herein may be implemented using computer software. If written in a programming language conforming to a recognized standard, sequences of instructions designed to implement the methods can be compiled for execution on a variety of hardware platforms and for interface to a variety of operating systems. In addition, embodiments of the present disclosure are not described with reference to any particular programming language. It will be appreciated that a variety of programming languages may be used to implement embodiments of the present disclosure.

It should be noted that the methods and apparatus described herein are not limited to use only with medical diagnostic imaging and treatment. In alternative embodiments, the methods and apparatus herein may be used in applications outside of the medical technology field, such as industrial imaging and non-destructive testing of materials. In such applications, for example, “treatment” may refer generally to the effectuation of an operation controlled by the treatment planning system, such as the application of a beam (e.g., radiation, acoustic, etc.) and “target” may refer to a non-anatomical object or area.

The above description of illustrated embodiments of the disclosure, including what is described in the Abstract, is not intended to be exhaustive or to limit the disclosure to the precise forms disclosed. While specific embodiments of, and examples for, the disclosure are described herein for illustrative purposes, various equivalent modifications are possible within the scope of the disclosure, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

In the foregoing specification, the disclosure has been described with reference to specific exemplary embodiments thereof. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense.

What is claimed is:

1. A method comprising:
imaging a first field of view (FOV) of a volume of interest (VOI) comprising a region of interest (ROI) of a patient from a first position with an imager;
imaging the first FOV of the VOI comprising the ROI of the patient from a second position with the imager;
receiving a first identification of a first portion of the imaged VOI from the first position designating the ROI to be imaged;
receiving a second identification of a second portion of the imaged VOI from the second position designating the ROI to be imaged;
in response to receiving the first identification, adjusting an aperture of a collimator of an imaging source to a second FOV corresponding to the ROI from the first position, the second FOV being different than the first FOV from the first position;
imaging the ROI of the patient from the first position using the second FOV;
in response to receiving the second identification, adjusting the aperture of the collimator of the imaging source to a third FOV corresponding to the ROI from the second position, the third FOV being different than the first FOV from the second position; and
imaging the ROI of the patient from the second position using the third FOV.

2. The method of claim 1, wherein the first identification of the first portion of the imaged VOI and the second identification of the second portion of the imaged VOI are based on a user selection of the ROI.

3. The method of claim 1, wherein the first identification of the first portion of the imaged VOI and the second identification of the second portion of the imaged VOI are based on an automatic identification of the ROI.

4. The method of claim 1, wherein the ROI in the VOI of the patient comprises at least one fiducial marker located within the VOI.

5. The method of claim 1, wherein the ROI in the VOI of the patient comprises an anatomical feature located within the VOI.

6. The method of claim 1, wherein adjusting the aperture of the imaging source comprises adjusting a size of the aperture to correspond to a size of the ROI.

7. The method of claim 1, wherein adjusting the aperture of the imaging source comprises adjusting a shape of the aperture.

8. The method of claim 1, wherein the aperture of the imaging source is adjusted while the kV imager moves from the first angle to the second angle.

9. A system comprising:
an x-ray imager comprising an x-ray imaging source and an x-ray detector, wherein the x-ray imaging source comprises a variable aperture collimator;
a processing device, operatively coupled to the x-ray imager, to:
image a first field of view (FOV) of a volume of interest (VOI) comprising a region of interest (ROI) of a patient from a first position with the x-ray imager;
image the first FOV of the VOI comprising the ROI of the patient from a second position with the x-ray imager;
receive a first identification of a first portion of the imaged VOI from the first position designating the ROI to be imaged;
receive a second identification of a second portion of the imaged VOI from the second position designating the ROI to be imaged;
in response to receiving the first identification, adjust the variable aperture of the x-ray imaging source to a second FOV corresponding to the ROI from the first position, the second FOV being different than the first FOV from the first position;
image the ROI of the patient from the first position using the second FOV;
in response to receiving the second identification, adjust the variable aperture of the x-ray imaging source to a third FOV corresponding to the ROI from the second position, the third FOV being different than the first FOV from the second position; and
image the ROI of the patient from the second position using the third FOV.

10. The system of claim 9, wherein the first identification of the first portion of the imaged VOI and the second identification of the second portion of the imaged VOI are based on a user selection of the ROI.

11. The system of claim 9, wherein the first identification of the first portion of the imaged VOI and the second identification of the second portion of the imaged VOI are based on an automatic identification of the ROI.

12. The system of claim 9, wherein the ROI in the VOI of the patient is at least one fiducial marker located within the VOI.

13. The system of claim 9, wherein the ROI in the VOI of the patient comprises an anatomical feature located within the VOI.

14. The system of claim 9, wherein adjusting the aperture of the x-ray imaging source comprises adjusting a size of the aperture to correspond to a size of the ROI.

15. The system of claim 9, wherein adjusting the aperture of the x-ray imaging source comprises adjusting a shape of the aperture.

16. The system of claim 9, wherein the aperture of the x-ray imaging source is adjusted while the imager moves from the first angle to the second angle.

17. The system of claim 9, wherein the x-ray imaging source comprises a kilovoltage (kV) x-ray imaging source.

18. The system of claim 9, wherein the x-ray imaging source comprises a megavoltage (MV) x-ray imaging source.

19. A non-transitory computer-readable storage medium having instructions that, when executed by a processing device, cause the processing device to:
image, from a first position with an x-ray imaging source, a volume of interest (VOI) using a first field of view (FOV), wherein the VOI comprises a region of interest (ROI) of a patient;
adjust an aperture of the x-ray imaging source to a second FOV corresponding to the ROI from the first position, the second FOV being different than the first FOV from the first position; and
image, from a second position with the x-ray imager, the VOI using the second FOV.

20. The non-transitory computer-readable storage medium of claim 19, wherein adjusting the aperture of the x-ray imaging source comprises adjusting a size of the aperture to correspond to a size of the ROI.

21. The non-transitory computer-readable storage medium of claim 19, wherein adjusting the aperture of the x-ray imaging source comprises adjusting a shape of the aperture to correspond to a shape of the ROI.

22. The non-transitory computer-readable storage medium of claim 19, wherein the aperture of the x-ray imaging source is adjusted while the x-ray imager moves from the first angle to the second angle.

23. A method comprising:
receiving an identification of an internal target region of a patient;
in response to receiving the first identification, adjusting an aperture of a collimator of an x-ray imaging source to a first field of view (FOV) corresponding to the internal target region;
periodically generating, by an x-ray imager, internal positional data about the internal target region using the first FOV;
continuously generating external positional data about external motion of the patient's body using an external sensor;
generating a correlation model between the position of the internal target region and the external sensor using the external positional data of the external sensor and the internal positional data of the internal target region;
predicting the position of the internal target region at some later time based on the correlation model; and
adjusting the aperture of the collimator of the x-ray imaging source to a second FOV corresponding to the predicted position of the internal target region.
